(19) United States
(12) Patent Application Publication
(10) Pub. No.: US 2014/0186263 A1
(43) Pub. Date: Jul. 3, 2014

(54) MALEIMIDE-FUNCTIONALIZED GOLD NANOPARTICLES

(71) Applicants: Ralf Schirrmacher, Montreal (CA); Jun Zhu, Montreal (CA); R. Bruce Lennox, Montreal (CA)

(72) Inventors: Ralf Schirrmacher, Montreal (CA); Jun Zhu, Montreal (CA); R. Bruce Lennox, Montreal (CA)

(73) Assignee: THE ROYAL INSTITUTION FOR THE ADVANCEMENT OF LEARNING/MCGILL UNIVERSITY, Montreal, QC (CA)

(21) Appl. No.: 14/122,505

(22) PCT Filed: May 31, 2012
(86) PCT No.: PCT/CA2012/000609

§ 371 Date: Feb. 10, 2014

Related U.S. Application Data
(60) Provisional application No. 61/491,638, filed on May 31, 2011.

(51) Int. Cl. A61K 51/06; A61K 51/12; A61K 47/48

(52) U.S. Cl. CPC A61K 51/065; A61K 47/48853; A61K 47/48215; A61K 51/1244; Y10S 977/777; B82Y 5/00
USPC 424/1.89; 548/403; 514/184; 548/431; 428/402; 977/777

(57) ABSTRACT

A maleimide-functionalized gold nanoparticle is described herein. More specifically, a maleimide-functionalized gold nanoparticle of formula (I): is described, wherein “n” and “m” are integers independently ranging from 1 to 100.

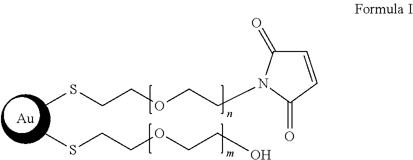

### CROSS-REFERENCE TO RELATED APPLICATIONS

The present application claims the benefit of priority from co-pending U.S. provisional application No. 61/491,638 filed on May 31, 2011, the contents of which are incorporated herein by reference in their entirety.

### FIELD

The present specification broadly relates to maleimide-functionalized gold nanoparticles. More specifically, but not exclusively the present specification relates to maleimide-functionalized gold nanoparticles for multimodal biological applications. The present specification also relates to a process for the preparation of maleimide-functionalized gold nanoparticles.

### BACKGROUND

Ligand-capped gold nanoparticles (L-AuNP) which consist of a small gold core and a monolayer of ligands have been extensively studied and characterized in the last two decades.^{[1,2]} Their high chemical stability, low toxicity, and the ease by which their surface can be designed, makes gold nanoparticles ideal bio-probes or vectors for probe/drug delivery in the application of biological diagnostics and therapeutics.^{[3-5]} Water soluble gold nanoparticles synthesized by the Turkevich method provide access to relatively large AuNP (>10 nm diameter).^{[6]}

Most AuNP studies relate to alkylthiolated protected gold nanoparticles, which usually have excellent stability in organic solvents and can be manipulated like many small organic molecules. In the Turkevich method, citrate serves both as the reducing reagent (to reduce HAuCl₄ in water) and as the capping ligand preventing the gold cores from aggregating. The size distribution of the citrate-capped AuNP can be controlled to vary between 5 to 147 nm by a selective choosing of the temperature, the ratio of gold to citrate, and the sequence of adding reagents.^{[7,8]} However, weak ionic interactions limit the stability of these AuNPs. Moreover, the NPs prepared using this method cannot easily be separated from water and re-dispersed in water after storage.

The use of tiopronin (N-(2-mercaptopropionyl)-glycine) as the protecting ligand to synthesize stable, thiolate-protected water-soluble AuNPs has been previously described.^{[9]} Unfortunately, the charge of the tiopronin-AuNPs prohibits their in-vivo application, as charged particles are susceptible to opsonization by proteins.^{[10]}

Polyethylene glycol (PEG) has been used to coat gold nanoparticles to reduce toxicity in vivo.^{[11,12]} The PEGylated species can reduce the interactions between proteins and nanoparticle surfaces through hydrophilicity and steric repulsion, thus reducing opsonization. It has been previously shown that PEGylated gold nanorods exhibit enhanced circulation time and demonstrate no cytotoxicity in vitro.^{[13]} Similarly, studies on the in vitro permeation of gold nanoparticles through rat skin and intestine, show that the larger the gold nanoparticles, the weaker their permeation ability.^{[14]} However, these studies were dealing with larger nanoparticles (>15 nm).

Mono-maleimide functionalized gold nanoparticles have been previously prepared by Hainfeld and coworkers.^{[15,16]} The maleimide containing monolayer protected nanoparticle (MPN) is prepared by mixing fluorescein-conjugated phosphine ligand protected MPNs with a 100 fold excess of N-methoxycarbonylmaleimide. This phosphine protected nanoparticle however suffers from low stability. It has to be stored at −4° C. and must be used at low temperatures. The low thermal stability reduces its potential for incubation conditions at 37° C. More importantly, it can degrade upon exposure to thiols, as thiols can undergo not only the desired Michael addition reaction but also the fast place-exchange reactions, replacing the weakly bound phosphine ligands.

Zhu et al. have prepared maleimide-MPNs which can dissolve in toluene, benzene, dichloromethane, and other organic solvents.^{[17]} They also studied cycloaddition reactions involving the maleimide-MPNs with dienes or nitrones to further modify the nanoparticles under harsh conditions, e.g. 96,000 atm.

Recently, Rodriguez et al synthesized maleimide-MPNs by ligand place-exchange of CTAB (cetyltrimethylammonium bromide) with amine-terminated PEG ligands, followed by subsequent treatment with a maleimide bearing bio-linker, such as sulfo-SMCC (sulfosuccinimidyl-4-[N-maleimidomethyl]cyclohexane-1-carboxylate) to prepare the desired NPs.^{[18]} A similar study was carried out by Oh et al via place-exchange of the citric acid protected NPs with a PEGylated ligand functionalized with thioctic acid and maleimide groups in both chain ends.^{[19]} However, both of these studies related to large nanoparticles which were limited by the size of the starting NPs

The present specification refers to a number of documents, the contents of which are herein incorporated by reference in their entirety.

### SUMMARY

The present specification broadly relates to maleimide-functionalized gold nanoparticles

In an embodiment, the present specification relates to a maleimide-functionalized gold nanoparticle of Formula I:

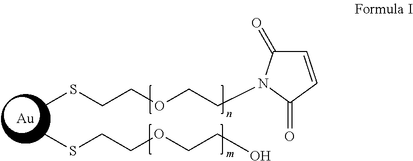

wherein “n” and “m” are integers independently ranging from 1 to 100.

In another embodiment, the present specification relates to a maleimide-functionalized gold nanoparticle of Formula II:

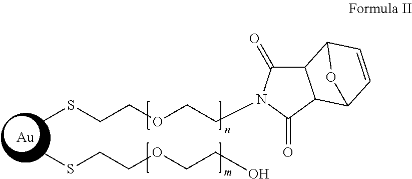

wherein “n” and “m” are integers independently ranging from 1 to 100.

In a further embodiment, the present specification relates to a maleimide-functionalized gold nanoparticle-based bioprobe of Formula III.

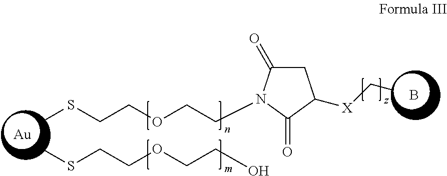

wherein “n” and “m” are integers independently ranging from 1 to 100;

X is selected from the group consisting of NR¹ and S;

R¹ is H or C₁₋₆alkyl;

B is a probe or a therapeutic agent (for example a biomolecular probe); and

“z” is an integer ranging from 0 to 5.

In a further embodiment, the present specification relates to a radiolabeled maleimide-functionalized gold nanoparticle of Formula IV:

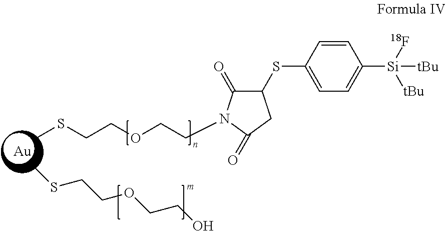

wherein “n” and “m” are integers independently ranging from 1 to 100.

In yet a further embodiment, the present specification relates to a furan protected maleimide-PEG-thiol having the formula:

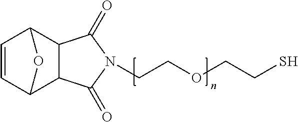

wherein “n” is an integer ranging from 1 to 100.

In an embodiment, the present specification relates to a maleimide-functionalized gold nanoparticle comprising from about 950 to about 2300 gold atoms.

In an embodiment, the present specification relates to a maleimide-functionalized gold nanoparticle comprising about 1000 gold atoms.

In an embodiment, the present specification relates to a maleimide-functionalized gold nanoparticle comprising a gold core having an average size ranging from 3 to 4 nm.

In an embodiment, the present specification relates to a maleimide-functionalized gold nanoparticle comprising a gold core having an average size of about 3.2 nm.

In an embodiment, the present specification relates to a maleimide-functionalized gold nanoparticle comprising maleimide-terminated ligands and PEG ligands. In an embodiment, the maleimide-terminated ligands are maleimide-terminated PEG ligands.

In an embodiment, the present specification relates to a maleimide-functionalized gold nanoparticle comprising maleimide-terminated ligands and PEG ligands, wherein the ratio of maleimide-terminated ligands to PEG ligands ranges from 1:1 to 1:3.

In an embodiment, the present specification relates to a water soluble maleimide-functionalized gold nanoparticle.

In an embodiment, the present specification relates to a process for preparing a maleimide-functionalized gold nanoparticle, the process comprising reacting PEGylated AuNPs with a furan-protected maleimide-PEG-thiol followed by the removal of the furan-protection group. In an embodiment, the present specification relates to a process for preparing a maleimide-functionalized gold nanoparticle of Formula I comprising heating a maleimide-functionalized gold nanoparticle of Formula II under conditions to form the maleimide-functionalized gold nanoparticle of Formula I. In an embodiment of the present specification, the reactive maleimide group is generated by heating the functionalized gold nanoparticle of Formula II in a suitable solvent or solvent mixture at a temperature ranging from about 50 to 150° C.

In an embodiment, the present specification relates to the use of a maleimide-functionalized gold nanoparticle for multimodal biological applications.

In an embodiment, the present specification relates to the use of a radiolabeled maleimide-functionalized gold nanoparticle of Formula IV as a radiotracer in spectroscopic imaging applications.

In an embodiment, the present specification relates to a method for delivering a therapeutic agent to a subject, the method comprising:

providing a nanoparticle of Formula I;

coupling the therapeutic agent to the nanoparticle of Formula I to form a nanoparticle-drug conjugate; and

administering to the subject the nanoparticle-drug conjugate.

In an embodiment, the present specification relates to a method for delivering a radioimmaging agent to a subject, the method comprising:

providing a nanoparticle of Formula I;

coupling the radioimmaging agent to the nanoparticle of Formula I to form a nanoparticle-radioimmaging agent conjugate; and

administering to the subject the nanoparticle-radioimmaging agent conjugate.

In an embodiment, the present specification relates to a method of using a radiolabeled maleimide-functionalized gold nanoparticle of Formula IV as a radiotracer for radioimmaging applications, the method comprising administering the radiolabeled maleimide-functionalized gold nanoparticle of Formula IV to a subject.

The foregoing and other objects, advantages and features of the present specification will become more apparent upon reading of the following non-restrictive description of illustrative embodiments thereof, given by way of example only with reference to the accompanying drawings/figures.

### BRIEF DESCRIPTION OF THE DRAWINGS/FIGURES

In the appended drawings/figures:

FIG. 1 is an illustration of the monitoring of the retro-Diels-Alder reaction of 6-AuNP, obtained in accordance with an embodiment of the present specification, by ¹H NMR spectroscopy (solvent D₂O); 6-AuNP at room temperature (FIG. 1a); 6-AuNP at 90° C. (FIG. 1b), 6-AuNP following heating at 90° C. over a period of 20 minutes (FIG. 1c); 6-AuNP following heating at 90° C. over a period of 30 minutes (FIG. 1d); 6-AuNP following heating at 90° C. over a period of 40 minutes (FIG. 1e); 6-AuNP following heating at 90° C. over a period of 50 minutes (FIG. 1f); 6-AuNP following heating at 90° C. over a period of 60 minutes (FIG. 1g); downward arrows and upward arrows corresponding to the resonance of the Diels-Alder adducts and the maleimide alkene protons, respectively.

FIG. 2 is a Transmission Electron Microscopy (TEM) image of 6-AuNP and Maleimide-AuNP (scale bar is 20 nm).

FIG. 3 is a Thermal Gravimetric Analysis (TGA) of 6-AuNP and maleimide-AuNP (programmed temperature rising speed 10° C./min.).

FIG. 4 is an illustration of the X-ray Photoelectron Spectrum (XPS) of maleimide-AuNP, N 1s and S 2p peaks are indicated.

FIG. 5 is an illustration of the fluorescent spectra for Rhodamine-MPN before and after treatment with I₂/KI (excited at 505 nm and emission at 530 nm).

FIG. 6 is an illustration of the ¹H NMR spectrum of a maleimide-AuNP; peaks at 5.82 ppm and 2.80 ppm correspond to the alkene protons of the maleimide and the protons α to the sulfur, respectively (solvent D₂O). As each maleimide-PEG ligand contains 2 alkene protons in addition to 2 protons α to sulfur and each PEG ligand contains 2 protons α to sulfur, the mole ratio of the maleimide-PEG-thiolated ligand.PEG-thiolated ligand can be determined from the integration as being 1:2.

FIG. 7 is an illustration of the radiolabeling of a maleimide-AuNP with [¹⁸F]-SiFA-SH. The upper portion depicts the radiolabeling reaction whereas the lower portion illustrates the time line for the maleimide-AuNP labeling. Broadly, the radiolabeling comprises: 1) the ¹⁸F labeling of SiFA-SH to produce [¹⁸F]SiFA-SH; 2) reacting [¹⁸]SiFA-SH with a maleimide-AuNP; and 3) purification of the [¹⁸F]SiFA-AuP by SEC.

FIG. 8 is an illustration of representative body and brain scan images of a micro PET scan following intravenous injection of a solution of [¹⁸F]-SiFA-AuNP in rats. Broadly, the images represent coronal (a), sagittal (b) and transverse planes (c); and brain scan images in coronal (d), sagittal (e) and transverse planes (f). Sum images t=60-120 min.

FIG. 9 is an illustration of the time-activity curves for cerebellum, brain and bone extracted from in vivo micro PET scans for up to 60 min post injection of [¹⁸F]-SiFA-AuNP. Each data point represents the average of three independent animal experiments. The bars represent errors which are expressed as SEM (n=3).

FIG. 10 is an illustration of the biodistribution of [¹⁸F]-SiFA-AuNP in selected tissues; injected dose for each gram of tissue (% ID/g) calculated from the ex vivo biodistribution (left) and zoom of the left figure to show the distribution of [¹⁸F]-SiFA-AuNP (right). The concentration of [¹⁸F]-SiFA-AuNP was highest in the kidneys (2%), liver (1.5%) and spleen (1.5%)

### DETAILED DESCRIPTION

### I. Definitions

In order to provide a clear and consistent understanding of the terms used in the present specification, a number of definitions are provided below. Moreover, unless defined otherwise, all technical and scientific terms as used herein have the same meaning as commonly understood by one of ordinary skill in the art to which this specification pertains.

Unless otherwise indicated, the definitions and embodiments described in this and other sections are intended to be applicable to all embodiments and aspects of the application herein described for which they are suitable as would be understood by a person skilled in the art.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

In embodiments comprising an “additional” or “second” component, such as an additional or second nanoparticle, the second component as used herein is chemically different from the other components or first component. A “third” component is different from the other, first, and second components, and further enumerated or “additional” components are similarly different.

As used in this specification and claim(s), the words “comprising” (and any form of comprising, such as “comprise” and “comprises”), having (and any form of having, such as “have” and “has”), “including” (arid any form of including, such as “include” and “includes”), or “containing” (and any form of containing, such as “contain” and “contains”), are inclusive and open-ended and do not exclude additional, unrecited elements or process steps.

The term “about” is used to indicate that a value includes an inherent variation of error for the device or the method being employed to determine the value.

As used herein, the term “alkyl” can be straight-chain or branched. This also applies if they carry substituents or occur as substituents on other residues, for example in alkoxy residues, alkoxycarbonyl residues or arylalkyl residues. Substituted alkyl residues can be substituted in any suitable position. Examples of alkyl residues containing from 1 to 18 carbon atoms are methyl, ethyl, propyl, butyl, pentyl, hexyl, heptyl, octyl, nonyl, decyl, undecyl, dodecyl, tetradecyl, hexadecyl and octadecyl, the n-isomers of all these residues, isopropyl, isobutyl, isopentyl, neopentyl, isohexyl, isodecyl, 3-methylpentyl, 2,3,4-trimethylhexyl, sec-butyl, tert-butyl, or tert-pentyl. A specific group of alkyl residues is formed by the residues methyl, ethyl, n-propyl, isopropyl, n-butyl, isobutyl, sec-butyl and tert-butyl.

As used herein, the term “lower alkyl” can be straight-chain or branched. This also applies if they carry substituents or occur as substituents on other residues, for example in alkoxy residues, alkoxycarbonyl residues or arylalkyl residues. Substituted alkyl residues can be substituted in any suitable position. Examples of lower alkyl residues containing from 1 to 6 carbon atoms are methyl, ethyl, propyl, isopropyl, butyl, isobutyl, tert-butyl, pentyl, isopentyl, neopentyl, and hexyl.

As used herein, the term “probe” means a chemical entity that probes, examines or tests. The entity will possess a site that interacts with a desired species, for example in solution and for example in a selective and detectable manner.

The term “biomolecular” probe as used herein refers to a probe that will interact with a biomolecule, such as a protein, peptide, amino acid, nucleic acid, polysaccharide, lipid, phospholipid and the like, for example, in solution and for example in a selective manner.

The term “therapeutic agent” as used herein refers to any agent or drug that has a pharmacological effect on a cell or a subject. Non-limiting examples of therapeutic agents include anti-inflammatory agents, antibiotics, antivirals, antineoplastic/antiangiogenic agents and antiproliferative agents.

The term “interact” as used herein means for one entity to associate with another entity in a manner such that the interaction is detectable.

The term “substituted” as used herein, means that a hydrogen radical of the designated moiety is replaced with the radical of a specified substituent, provided that the substitution results in a stable or chemically feasible compound. Non-limiting examples of substituents include halogen (F, Cl, Br, or I) for example F, and C₁₋₄alkyl.

As used herein, a gold nanoparticle can be depicted using either

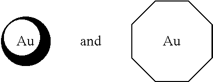

Both are used interchangeably throughout the present specification.

As used herein, the expression “multimodal” means to have more than one mode of operation or function, for example, for the delivery of more than one therapeutic agent and/or for probing more than one biomolecule and/or for the delivery of more than one radioimmaging agent.

The term “suitable” as used herein means that the selection of the particular compound or conditions would depend on the specific synthetic manipulation to be performed, and the identity of the molecule(s) to be transformed, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The expression “proceed to a sufficient extent” as used herein with reference to the reactions or process steps disclosed herein means that the reactions or process steps proceed to an extent that conversion of the starting material or substrate to product is maximized. Conversion may be maximized when greater than about 5, 10, 15, 20, 25, 30, 35, 40, 45, 50, 55, 60, 65, 70, 75, 80, 85, 90, 95 or 99% of the starling material or substrate is converted to product.

The term “subject” as used herein means a mammal. Typically the subject is a human.

### II. Gold Nanoparticles and Compositions and Uses Thereof

The chemical properties of AuNPs are principally determined by the surrounding ligands. A variety of functional alkyl-thiolated protected AuNPs can be prepared via direct synthesis, place exchange reactions or post-synthesis reactions. The first two methods require tedious and time-consuming work to prepare each individual thiol. In addition, these methods tend to suffer from low thiol efficiency, especially for the place-exchange reactions which require a large excess of thiol to maleimide ratios to effectively replace the existing ligands. In an embodiment of the present specification, ratios of PEG-thiol/furan-protected maleimide-PEG-thiol in excess of 1:1 for example 2:1, 3:1, 4:1, 5:1, 6:1, 7:1, 8:1, 9:1 10:1, 20:1 or higher were used to effectively replace the existing ligands.

The development of a functional AuNP which could be treated as analogous to common organic molecules is an important aspect for its application in a functional platform. This template AuNP should contain one or more active end-groups which can be further transformed via commonly used organic reactions, such as nucleophilic substitution reactions, nucleophilic addition reactions. Fisher esterification reactions and/or cycloaddition reactions, as a means to introduce new functional groups. Moreover, a template for use to post-modify AuNPs optimally has the following characteristics: it should effectively undergo organic reactions under mild conditions; the functional groups on the AuNP should react selectively with the incoming molecules; and the introduced molecules should require only little or no pre-modification steps before the installation.

The maleimide-thiol reaction is a popular reaction used by biochemists to effectively couple a peptide of interest to a substrate Moreover, as a variety of biomolecules are naturally bearing thiol or amino groups, a maleimide functionalized nanoparticle is a promising template for use as a carrier for drug delivery or therapeutic diagnostics.

In an embodiment, the present specification relates to an economic, efficient and reproducible method for preparing stable, water-soluble, maleimide-terminated thiolate protected gold nanoparticles.

In an embodiment, the present specification relates to the preparation of size controlled PEGylated gold nanoparticles with an average size ranging between about 2-10 nm, following a modified Brust method.^{[20]} Subsequently, these PEGylated AuNPs were modified with a furan-protected maleimide-PEG-thiol. The resulting furan-protected maleimide-monolayer protected nanoparticle (MPN) has excellent thermal and temporal stability and the reactive maleimide group can be generated via a retro-Diels-Alder reaction simply by heating at 100° C. over a period of two hours.

¹H-NMR spectroscopy represents a powerful tool to examine the structure and composition of the MPN monolayers.^{[21,22]} The resonance peaks of the MPN monolayers are characteristically broad mainly due to spin-spin relaxation (T₂) broadening. The dense packing of the ligand chains close to the gold core results in a solid-like character due to dipolar interactions which translate into a fast spin relaxation. The short relaxation time results in a broadened and less intense signal. However, it is still possible to assign the peaks of the ¹H-NMR spectrum of the MPN monolayers from their chemical shifts and gain important structural information. More importantly, the absence of sharp peaks is an important characteristic of the purity of the MPN monolayers.

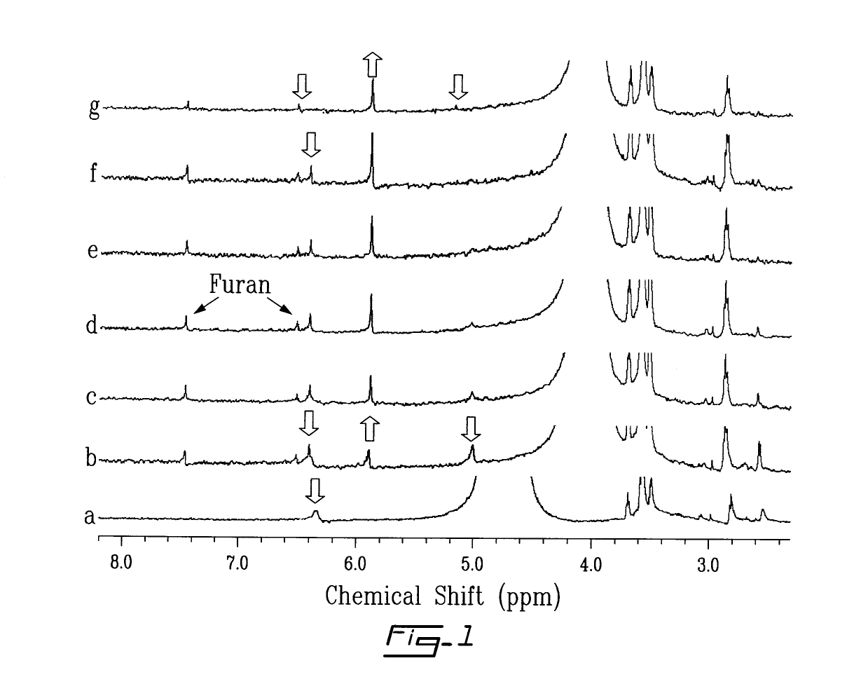

The maleimide-NPs prepared in accordance with an embodiment of the present specification possess excellent stability and can be purified by washing and drying using regular organic work-up methods. ¹H-NMR spectroscopy was applied to monitor the retro-Diels-Alder reaction (for liberation of the maleimide) and was used to verify the purity of MPNs (FIG. 1).

Results demonstrated that the maleimide-NP can effectively react with amine or thiol model compounds via a Michael addition reaction which is indicative that the maleimide-NPs can be used as a platform to prepare mono-modular or multi-modular probes by applying different combinations of thiol/amino containing probe molecules. Moreover, these maleimide-NPs can also be used as drug delivery vehicles by linking a drug to the maleimide group. In one embodiment, the drug comprises a thiol and/or amino function that can be used for linking the drug to the maleimide NPs. In an embodiment, this drug-linking is accomplished using a thiol/amino containing linker. Furthermore, these maleimide-NPs can also be used as radioimmaging delivery vehicles by linking a radioimmaging agent or its precursor to the maleimide group.

The preparation of PEGylated MPN, in accordance with an embodiment of the present specification, is illustrated hereinbelow in Scheme 1.

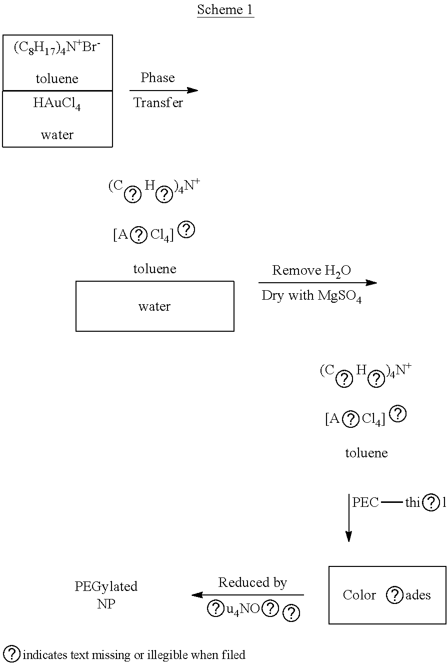

The synthesis of maleimide-PEG thiol (6) and maleimide-functionalized gold NPs (Maleimide-AuNP) in accordance with an embodiment of the present specification is illustrated hereinbelow in Scheme 2.

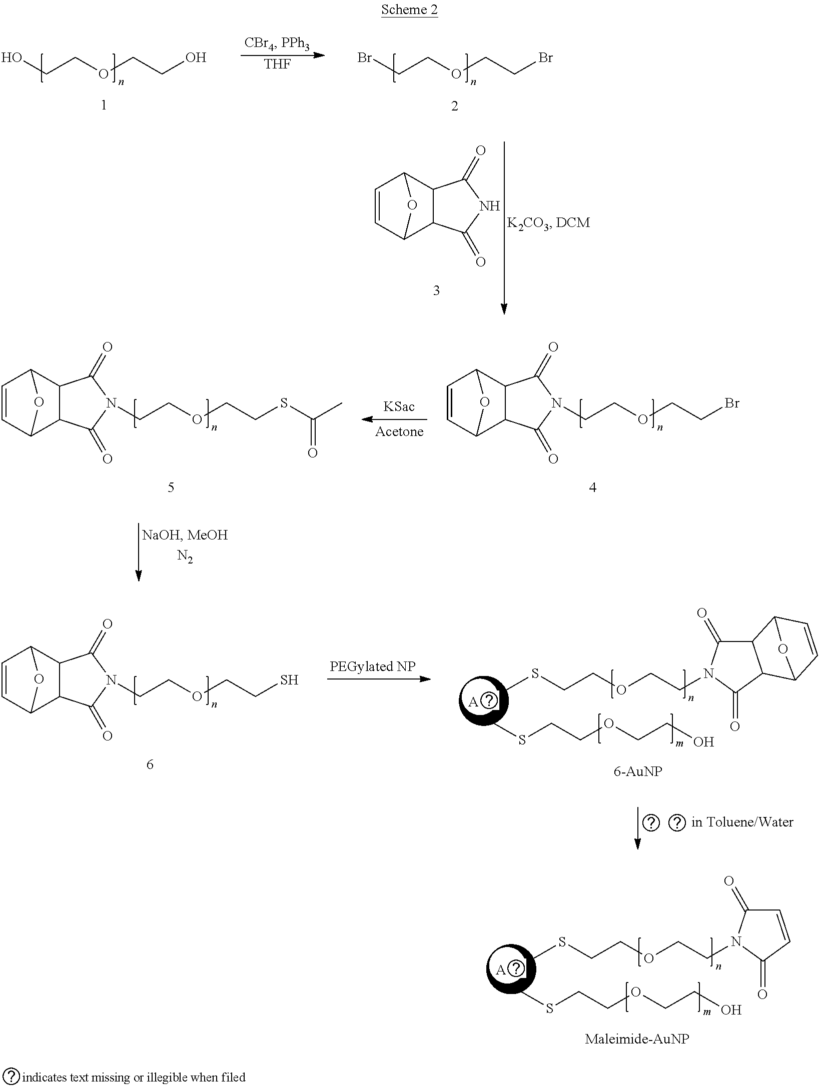

The maleimide-functionalized gold NP could not be prepared by direct synthesis as a strong reducing reagent is usually applied during the reduction of Au(I) to Au(0). The desired maleimide-functionalized gold NP could also not be directly prepared via the commonly used place-exchange reaction with maleimide-PEG-thiol, because maleimide is a good thiol-scavenger and the maleimide-tethered thiol is likely to undergo a Michael addition reaction with itself. To circumvent these problems, a furan protected maleimide-PEG-thiol (6) was synthesized so that it could be place-exchanged onto a PEGylated AuNP. The reactive maleimide group could then be generated by heating. Schemes 1 and 2 depict the modified Brust method used for the preparation of the PEGylated nanoparticle^{[20]} (Scheme 1) and the preparation of the maleimide-functionalized gold NP (Scheme 2), respectively.

In an embodiment of the present specification, triethylene glycol (n=2) was used.

In an embodiment of the present specification, the PEGylated AuNPs were prepared as follows. Hydrogen tetrachloroaurate (HAuCl₄) or potassium tetrabromoaurate (KAuBr₄) was dissolved in Milli-Q water and then mixed with tetraoctylammonium bromide (TOAB) in toluene. The contents were vigorously stirred in order to facilitate the phase transfer of the Au(III) into the toluene layer. The organic layer was separated and dried with MgSO₄ in order to remove any residual water and then cooled to 0° C. in an ice bath. Triethylene glycol-thiol “(TEG)-thiol”, as a non-limiting example of a pegylated thiol, was added to the solution via a volumetric pipette and allowed to stir for about 10 minutes. The observed dark orange solution faded with time. When the thiol/gold molar ratio exceeded 2:1, the solution became clear and colorless. Different sizes of AuNP can be obtained by varying the gold/thiol ratio. A fresh solution of tetrabutylammonium borohydride was then quickly added (in about 5 seconds) to a rapidly stirring toluene solution. The solution instantly turned black. The TEGylated NPs began to precipitate from the toluene after about 2 hours. After stirring the mixture overnight (˜12 hours), 30 mL of Milli-Q water was added to extract the TEGylated AuNP. The TEGylated AuNPs were purified by washing with toluene/acetonitrile or dialysis. The resulting pure AuNP sample was assessed by ¹H NMR spectroscopy, and showed no sign of free ligand. Unlike previously reported PEGylated NPs prepared from place-exchange reactions with citrate-nanoparticles or CTAB-nanoparticles, this TEGylated MPN has a smaller size, e.g. 3 nm, good stability, and could be easily prepared, purified, and characterized. It could also be repeatedly dried and readily redissolved in water.

The protected maleimide-PEG-thiol (6) was prepared as described hereinabove in Scheme 2. 3,6-Endoxo-Δ⁴-tetrahydrophthalimide (3) was reacted with excess dibromo-substituted PEG via a S_{N}2 reaction to generate the mono-substituted compound 4. The remaining bromide end-group of compound 4 was converted to the corresponding thioacetate 5 by reaction with KSAc/Acetone. Subsequent hydrolysis yielded the protected maleimide-PEG-thiol 6 in an overall yield of 30% (calculated starting from compound 3). The furan protected maleimide-PEG-thiol 6 was then incorporated into the previously prepared PEGylated AuNPs following a standard place-exchange reaction by mixing thiol 6/pegylated AuNP (mole ratio of 1:1) over a period of one hour to generate the mixed ligand 6-AuNP. The purity of 6-AuNP, following a purification step, was confirmed by ¹H-NMR spectroscopy (FIG. 1). The key resonances include the broad resonance at 6.35 ppm due to the alkene protons and those at 2.50 ppm due to the fused protons from the Diels-Alder adduct (FIG. 1a). The resonance of the methylene bridgehead protons was merged with the solvent peak while at room temperature. However, at an elevated temperature of 90° C., the solvent peak shifts downfield and the signal at 4.95 ppm can be assigned to the methylene bridgehead protons (FIG. 1b)

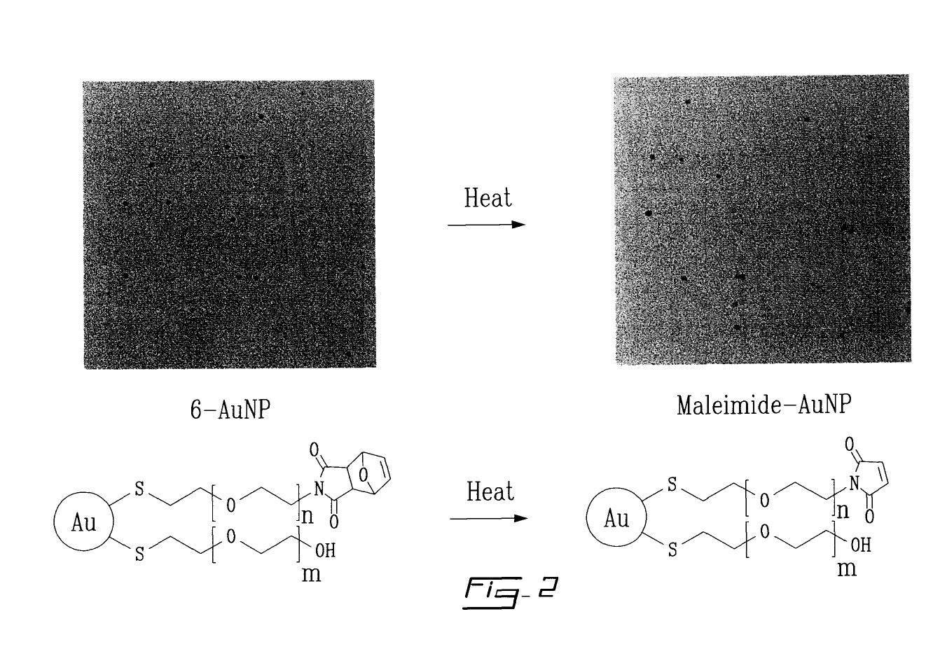

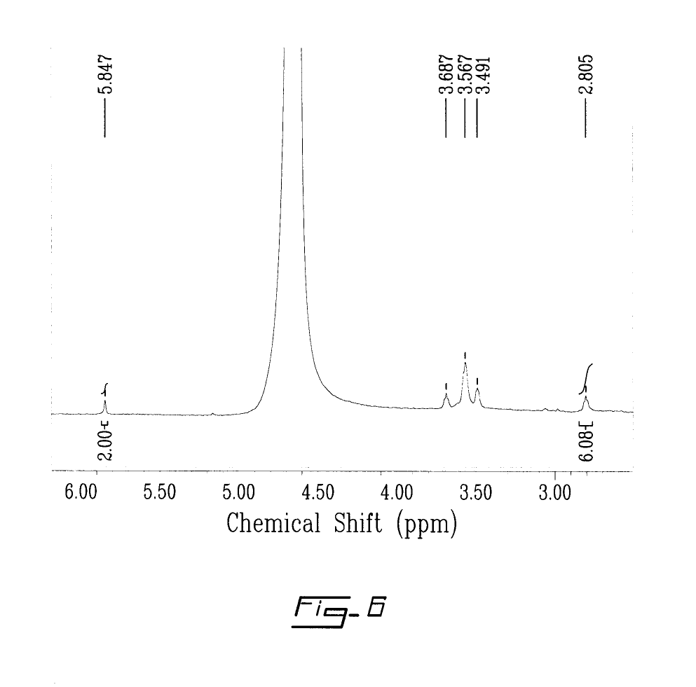

The maleimide terminated gold nanoparticles (maleimide-AuNPs) were readily generated at elevated temperatures from the corresponding purified furan protected maleimide-MPN (6-AuNP). FIG. 1 illustrates the monitoring of the retro-Diels-Alder reaction by varied-temperature ¹H-NMR spectroscopy. The broad peak at 5.82 ppm (indicated by the upward arrow; FIG. 1g) corresponds to the alkene protons of the maleimide whereas the peaks at 7.45 ppm and 6.50 ppm are attributed to the free furan liberated from the retro-Diels-Alder reaction (the furan peaks eventually disappear after heating for 90 min). The maleimide end-group started to be produced while heating and was completely recovered following heating at 90° C. over a period of one hour. The product maleimide-AuNPs were readily soluble in water following drying and could be re-dissolved for several cycles. The molar ratio of maleimide-PEG-thiolated ligand to PEGylated ligand was determined from the integration of the ¹H-NMR spectrum of maleimide-AuNP to be 1:2 (FIG. 6). TEM images of 6-AuNP before and after heating (maleimide-AuNP) showed a small increase of the core size and wider dispersion from 3.0±0.5 nm to 3.2±0.8 nm (FIG. 2). This slight increase in core size was due to small quantities of ligand which were liberated as disulfide during heating.

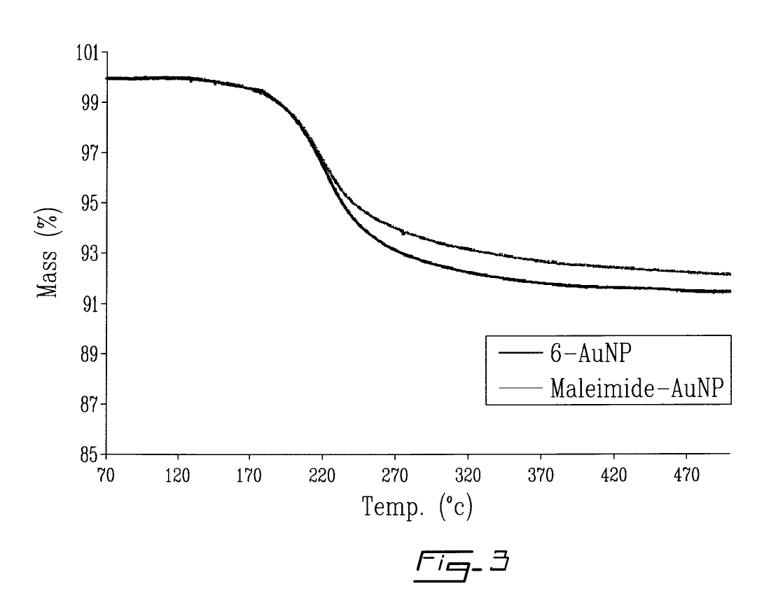

Thermal gravimetric analysis (TGA) provided direct information on the quantity of organic components on the Au NP (FIG. 3). The total mass loss of the furan-masked AuNP (6-AuNP) was determined to be 8.6%. For the maleimide-protected AuNP, the total mass loss was 8.0%. Ligands were removed from the gold NP surface starting at about 100° C. The additional mass loss for 6-AuNP accounts for the loss of furan from the retro Diels-Alder reaction.

Analysis of the data derived from TEM, NMR, and TGA experiments provided for an estimation of the composition of the AuNPs. Previous studies have indicated that the gold core has a truncated octahedron shape.^{[23]} To simplify the calculation, it was assumed that the maleimide-AuNP has a spherical shape. The average diameter of the maleimide-AuNP core could be derived from the planar sphere projection (from TEM derived to be 3.2 nm). It was also assumed that the gold core has the same density as bulk gold, the average number of gold atoms per AuNP is thus ca. 1000, using the formula:^{[24]}

$$N = \frac{πρ {d}^{3}}{6 {M}_{Au}} {N}_{A} ,$$

where

N=number of atoms per nanoparticle;

ρ=density of face centered cubic (fcc) gold=19.3 g/cm³,

d=average diameter of the nanoparticles;

M_{Au}=mole atomic weight of gold=196.9665 g/mol;

N_{A}=Avogadro constant.

Thus the total number of ligands per gold nanoparticle can be calculated from the weight percentage of the organic portion of the MPN In an embodiment of the present specification, the mixed ligand maleimide-AuNP is capped with two different ligands (e.g. maleimide-TEG-thiol ligands and TEGylated thiol ligands), and the average protecting ligands number is determined to be 90, using:

$${N}_{L} = ω \frac{{NM}_{Au} / ( 1 - ω )}{{M}_{L 1} ϕ + {M}_{L 2} ( 1 - ϕ ) ,}$$

where,

N_{L}=total number of ligands per nanoparticle;

ω=percentage of mass loss due to the protecting ligands;

M_{L1}=molecular weight of the maleimide-TEG-thiol ligands (C₁₀H₁₄O₄NS=244.0643 g/mol);

M_{L2}=molecular weight of the TEGylated thiol ligands (C₆H₁₃O₃S=165.0585 g/mol);

φ=the molar percentage of the maleimide ligand.

The area per grafted ligand is thus 0.35 nm², recognizing that this composition is based on using a gold core having a size of 3.2 nm. The total mass of organic/gold particle was determined, providing for the determination of the relative contribution of the two organic species (ligands) to this total mass. This yielded a weighted average number of molecules per particle. Since the average surface area of the gold core is known, the area occupied per grafted ligand could be determined. An area of 0.35 nm² is consistent with a moderately bulky thiol ligand. The simplified formula of the maleimide-AuNP can then be represented as Au₁₀₀₀(TEG)₆₀(Maleimide)₃₀.

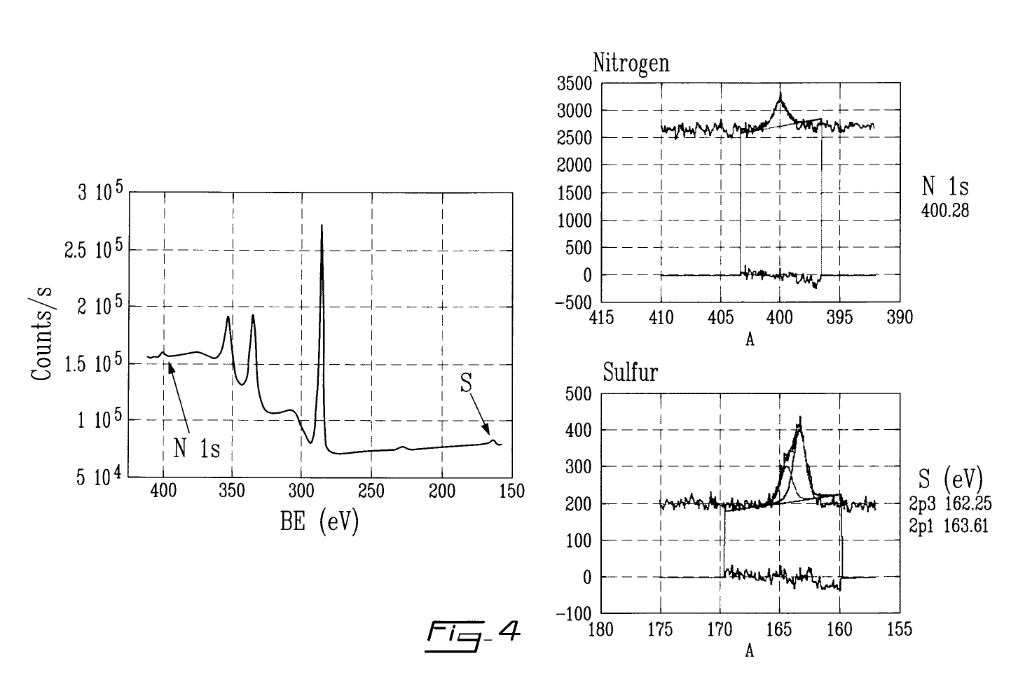

X-ray photoelectron spectra (XPS) of the maleimide AuNP confirm the presence of the maleimide group (FIG. 4). The nitrogen N1s BE value of 400.28 eV indicates the presence of the maleimide on the AuNP surface. The sulfur/nitrogen (S:N) ratio was 7:3, which was slightly lower than the calculated ratio of 9:3 (from the ¹H-NMR integration). This discrepancy likely arises from an attenuation of the S signal relative to the N signal in the XPS experiment, given that the S is directly bonded to the gold surface and is thus buried relative to the N.

Unlike small organic molecules, the reactivity of functional ligands on NPs might be quite different because of the steric environment of the NP ligands' corona. It was thus important to assess the reactivity and availability of the maleimide end groups of the maleimide-AuNPs. Maleimide-AuNPs were thus mixed with a large excess of furan (e.g. >2000 molar ratio) in aqueous solution over a period exceeding of a week. As expected, both exo- and endo-products were formed after the forward Diels-Alder reaction in a ratio of 5:4. This result confirmed that the maleimide end group in the maleimide-AuNP is freely accessible and reactive. Further modification of the maleimide-AuNP via Michael addition reaction would thus be facilitated.

### EXPERIMENTAL

### EXAMPLE 1

The reactive maleimide end groups of the maleimide-AuNPs were shown to readily react with amines or thiols via the Michael addition reaction (Scheme 3). Cysteine modified biomolecules, such as for example cystein-derivatized octreotate, represent a large class of bioprobe/precursors. The model Michael addition reaction between a maleimide-AuNP and cysteine was studied by ¹H-NMR spectroscopy. Although the new product peaks overlap with the resonance signal of the PEGylated ligands, the disappearance of the maleimide alkene proton peaks indicates that the Michael addition reaction proceeds under these conditions.

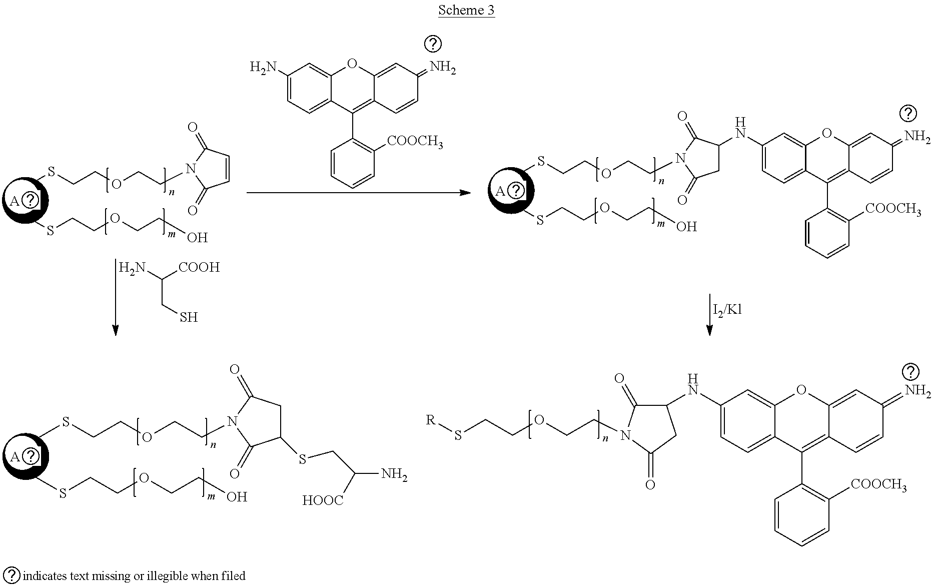

### EXAMPLE 2

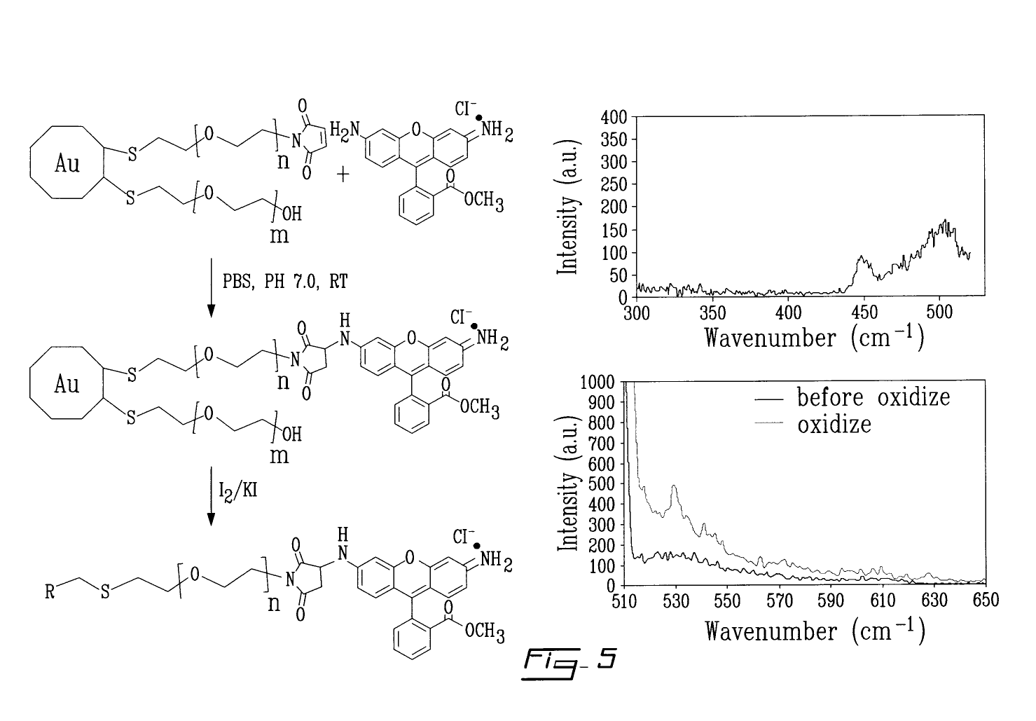

The conjugation of the amino dye, rhodamine 123 (Scheme 3), was assessed by fluorescence spectroscopy which is more sensitive than NMR spectroscopy. Rhodamine 123 was mixed with maleimide-AuNP in an aqueous solution over a period of 1 h. The resulting rhodamine-AuNPs were purified by washing with an ethyl acetate:ethanol solvent mixture to remove any free rhodamine 123 until no fluorescence could be detected in the organic layer Because the fluorescence of this fluorophore-coupled NP was quenched by the gold core (FIG. 5)^{[25]}, cleavage of the ligand from the AuNP surface was necessary. Indeed, following I₂ cleavage^{[26]}, the fluorescence reappeared, indicating that the Michael addition reaction of Rhodamine 123 had occurred. Following cleavage from the gold AuNP, a mixture of compounds is obtained, comprising the 2 homodimers (i.e. the disulfide based on the rhodamine-maleimide-PEG-thiol ligand and the disulfide based on the PEG-thiol ligand) and the disulfide dimer based on both the rhodamine-maleimide-PEG-thiol ligand and PEG-thiol ligand.

### EXAMPLE 3

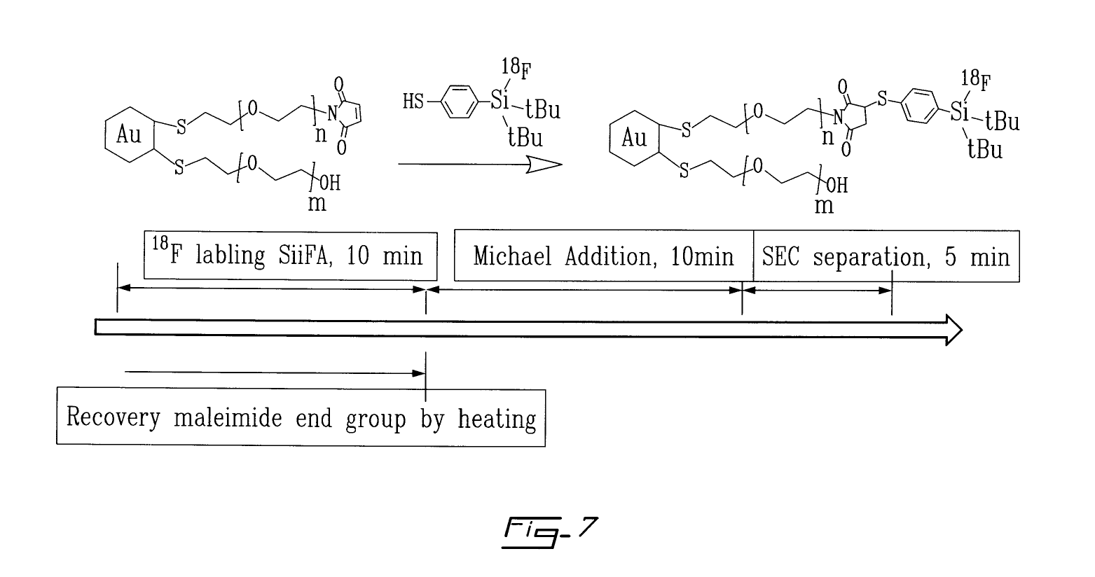

The radiolabeling of a maleimide-AuNP with ¹⁸F-labeled 4-(di-tert-butylfluorosilanyl)benzenethiol ([¹⁸F]-SiFA-SH) for Positron Emission Tomography (PET) was performed as follows (FIG. 7): 4 (di.tert-Butylflurosilyl)benzenethiol (SiFA-SH) was prepared and [¹⁸F]-SiFA-SH labeling was subsequently performed following a known literature procedure.^{[27]} SiFA-SH was labeled via a fast isotope exchange reaction using [¹⁸F]F [¹⁸F]-SiFA-SH was then incorporated into the maleimide-AuNP via a Michael addition reaction. The resulting [¹⁸F]-SiFA-AuNP was then purified through a NAP-10 size exclusion column. Due to the characteristic black color of AuNPs, the pure [¹⁸F]-SiFA-AuNP fraction could be easily separated and collected in a radiochemical yield of 60%. Its purity was confirmed by radio-TLC. A control labeling experiment was also carried out using [¹⁸F]-SiFA-OH and a maleimide-AuNP following the procedure illustrated in FIG. 7. Following separation of the ligand, there was no radioactivity observed on the AuNP which confirms that [¹⁸F]-SiFA-SH was attached to the NP surface via the desired Michael Addition reaction instead of physical adsorption.

### EXAMPLE 4

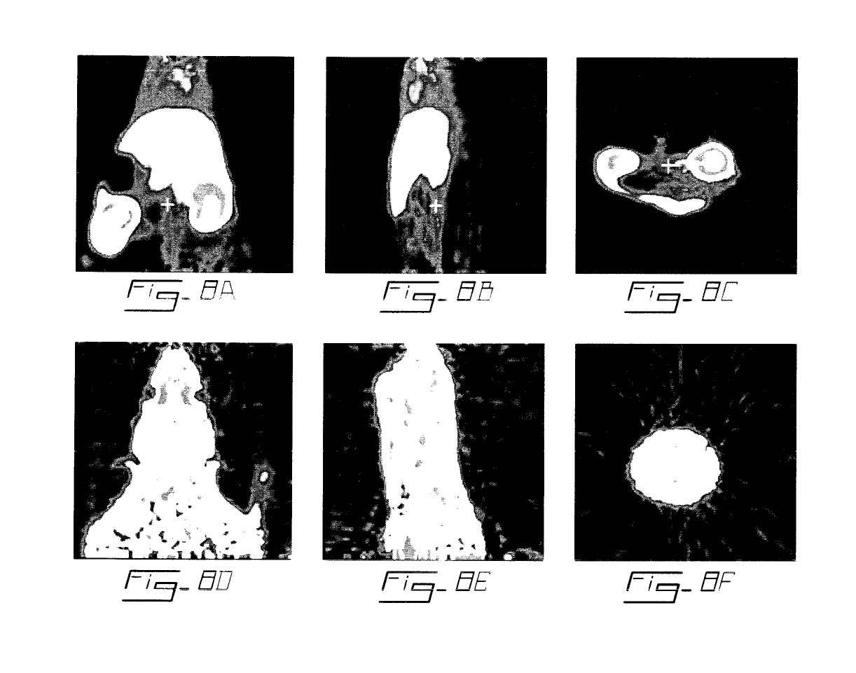

To evaluate the bio-distribution of the AuNPs, [¹⁸F] SiFA-AuNP (12 MBq) were injected into healthy rats (n=3) and evaluated by micro-PET. Representative body and brain scan images of a micro-PET scan following intravenous injection of a solution of [¹⁸F]-SiFA-AuNP are illustrated in FIG. 8. Radioactivity was found in the kidney, liver and spleen. One prominent result from these studies was that this small [¹⁸F]-SiFA-AuNP was able to cross the blood brain barrier (BBB) as shown in FIGS. 8d, e and f, even without specific modification of the AuNP surface with membrane penetrating peptides or proteins. Of note that [¹⁸F]-SiFA-SH alone does not cross the BBB.

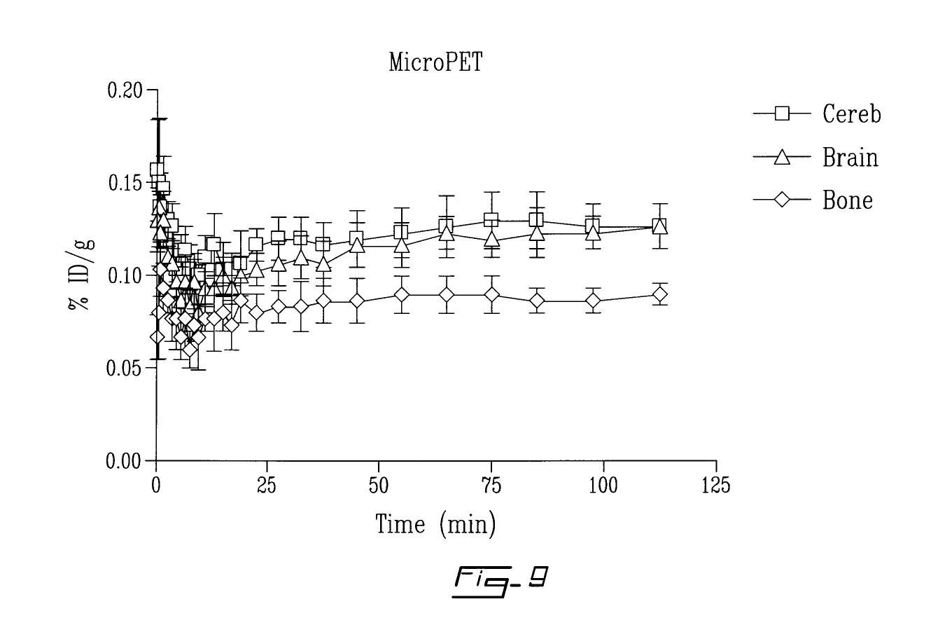

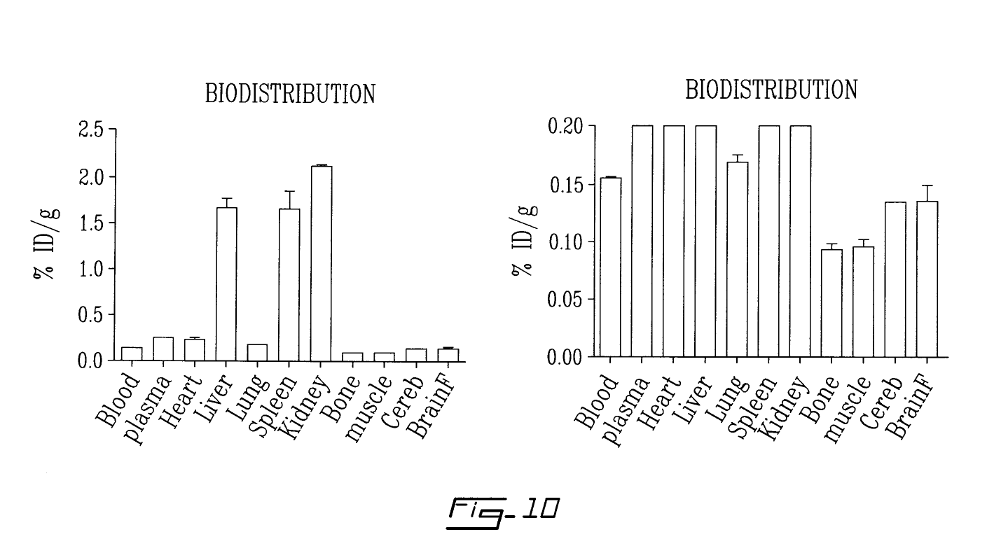

The time-activity curves obtained from the PET images depicting the radioactivity uptake in the cerebellum and the brain (n=3), using bone radioactivity as a region of reference, are illustrated in FIG. 9 These curves illustrate that AuNPs start to accumulate in the cerebellum and brain at 25-120 min. Ex vivo biodistribution studies were also carried out to confirm the bio-distribution data of [¹⁸F]-SiFA-AuNP in different organs 2 h following injection as obtained by micro-PET. The biodistribution data is illustrated in FIG. 10 and is expressed as the percentage of injected dose for each gram of tissue (% ID/g). The bio-distribution data were derived from an average of three rats (n=3). The bio-distribution results were in agreement with the results obtained from the PET images. The % ID/g value for brain and cerebellum is about 0.13%, about 50% higher than for bone uptake (0.08%), making the AuNP a promising delivery platform for the development of new PET tracers or radio-therapeutic agents. As there were no specific binding moieties conjugated to the AuNPs, the [¹⁸F]-SiFA-AuNP was homogenously distributed in the brain and no aggregation could be observed. The use of an ¹⁸F-labeled AuNP for PET studies using a large NP (e.g. 13 nm) was recently reported.^{[28]} However, the ¹⁸F-labeled AuNP could not be purified making an exact determination of its composition very difficult. Furthermore, larger AuNPs were reported as being unable to effectively cross the BBB.

The kidneys had the highest concentration of [¹⁸F]-SiFA-AuNP (2%), indicative of a renal clearance of the [¹⁸F]SiFA-AuNPs (FIG. 10). Moreover, as the liver and spleen also had high concentrations of [¹⁸F]-SiFA-AuNP (1.5%), respective hepatic and reticuloendothelial system (RES) clearances are also suggested. In view of the short half-life of ¹⁸F (t_{1/2}=110 min), a long term study of the clearance process was not performed.

[¹⁸F]-SiFA-SH was shown to effectively react with the water soluble maleimide-AuNPs of the present disclosure to generate radioactively labeled [¹⁸F]-SiFA-AuNPs. In an embodiment, these water soluble maleimide-AuNPs comprise a gold core having a size ranging from 3 to 4 nm. The labeled SiFA-AuNPs were readily purified by size exclusion column chromatography The radiolabeling was accomplished via a Michael addition reaction and no physical adsorption was observed when using [¹⁸F]-SiFA-OH which does not undergo Michael additions. The [¹⁸F]-SiFA-AuNPs were able to cross the blood brain barrier (BBB) as illustrated by small animal micro-PET studies as well as by ex vivo biodistribution data.

### EXAMPLE 5

Radiosynthesis of [¹⁸F]-SiFA-SH: [¹⁸F]fluoride/H₂[¹⁸O]O (30 mCi) was passed through a QMA cartridge preconditioned with 10 mL of 0.5M K₂CO₃ and 10 mL of H₂O. The trapped ¹⁸F was then eluted with 1 mL of a stock solution containing 75 mg (0.20 mmol) Kryptofix2.2.2 and 0.10 mmol potassium oxalate in 10 mL of 96/4 acetonitrile/H₂O solution. The residual water was azeotropically dried with anhydrous acetonitrile (MeCN) at 105° C. [¹⁸F]F cryptate was dissolved in 0.3 mL of anhydrous MeCN. SiFA-SH (40 μg, 148 nmol) in 0.2 mL of dry MeCN was added and reacted at room temperature for 10 min without stirring. This [¹⁸F]SiFA-SH/MeCN (20 mCi) solution was used for Maleimide-AuNP conjugation without further purification.

Reaction of [¹⁸F]-SiFA-SH with Maleimide-AuNP: 2 mL of the stock Maleimide-AuNP solution (55 mg in 25 mL water) was lyophilized and re-dissolved in 1 mL of phosphate buffer (0.1 M, pH 7.2). The maleimide reactive part was recovered by heating the solution at 100° C. for 2 h. To this solution was subsequently added [¹⁸F]SiFA-SH (20 mCi; obtained as described above) and reacted at room temperature for 10 min without stirring. The ¹⁸F-labeled AuNP was purified using a Nap-10 column (equilibration buffer 0.1 M PBS, pH 7.2). The black colored fraction was collected and the purity of the product was confirmed by TLC. The radiochemical yield was approximately 60% (12 mCi).

Biodistribution of [¹⁸F]-AuNP in Rats by small animal PET: SD (Sprague-Dawley) Rats for in vivo PET studies were housed in a 12 h light/dark cycle at 21° C. with access to food and water ad libitum. Treatment was in accordance with the Guide to the Care and Use of Experimental Animals (Ed2) of the Canadian Council on Animal Care. The Micro-PET imaging protocol was approved by the Animal Care Committee of McGill University (Montreal, Canada). The rats were kept under isoflurane anaesthesia for the injection of the radiotracer. Respiration rate, heart rate and body temperature were monitored throughout the scan (Biopac sytems MP150, Goleta, Calif., USA). 12-15 MBq of [¹⁸F]-AuNP were intravenously administered via the lateral tail vein. Whole body data were acquired for 1 h and brain data were acquired for 2 h using a Concord MicroPET R4 small animal tomograph. All images were reconstructed using filtered back projection after applying normalized scatter correction for attenuation and radioactive decay. The PET images were analyzed using ASIPRO software (Concorde Microsystems). The time-activity curves were obtained from regions of interest in heart and brain using bone and muscle as the references.

The SD rats were euthanized 2 h post-injection of the radiotracers. The organs were removed, rinsed with physiological saline, blotted dry and placed in pre-weighted tubes. The radioactivity in each tube was counted in a gamma counter (Cobra II, Packard Instruments) and used to calculate the percentage of the injected dose per gram of tissue (% ID/g) for each organ. The means from three rats are reported.

It is to be understood that the specification is not limited in its application to the details of construction and parts as described hereinabove. The specification is capable of other embodiments and of being practiced in various ways. It is also understood that the phraseology or terminology used herein is for the purpose of description and not limitation. Hence, although the present invention has been described hereinabove by way of illustrative embodiments thereof, it can be modified, without departing from the spirit, scope and nature of the subject disclosure as defined in the appended claims.

### REFERENCES

1. Cheon, J.; Lee, J. Acc Chem. Res., 2008, 41, 1630-1640.
2. Daniel, M. C.; Astruc, D. Chem, Rev., 2004, 104, 293-346.
3. (a) Rosi, N. L.; Mirkin, C. A. Chem. Rev., 2005, 105, 1547-1562. (b) Rosi, N L.; Giljohann, D. A.; Thaxton, C. S.; Lytton-Jean, A. K. R.; Han, M. S., and Mirkin, C. A. Science, 2006, 312, 1027-1030.
4. (a) Sonnichsen, C.; Reinhard, B. M.; Liphardt, J.; Alivisatos, A. P., Nature Biotechnology, 2005, 23, 741-745. (b) Reinhard, B. M.; Siu, M.; Agarwal, H.; Alivisatos, A. P.; Liphardt, J., Nano Letters, 2005, 5, 2246-2252.
5. El-Sayed, I. H.; Huang, X.; and El-Sayed, M. A. Nano Lett., 2005, 5, 829-834.
6. Turkevich, J.; Stevenson, P. C.; and Hillier, J. Discuss Faraday Soc., 1951, 11, 55-75.
7. Frens, G. Nature: Phys. Sci., 1973, 241, 20-22.
8. (a) Lo, C. K.; Xiao, D.; and Choi, M. M. F. J. Mater. Chem., 2007, 17, 2418-2427, (b) Patil, V.; Mayya, K. S.; and Sastry, M. Langmuir, 1999, 15, 6587-6590. (c) Yonezawa, T.; Kunitake, T. Colloid Surf. A.: Physicochem Eng Asp. 1999, 149, 193-199.
9. Templeton, A. C.; Chen, S.; Gross, S. M.; and Murray, R. W. Langmuir, 1999, 15, 66-76.
10. Owens, D. E. III; and Peppas, N. A. Int. J. Pharm., 2006, 307, 93-102.
11.Simpson, C. A.; Huffman, B. J.; Gerdon, A. E.; and Cliffel, D. A. Chem Res. Toxicol, 2010, 23, 1608-1616.
12. Liu, Y.; Shipton, M. K.; Ryan, J.; Kaufman, E. D.; Franzen, S.; and Feldheim, D. L. Anal. Chem., 2007, 79, 2221-2229.
13. Niidome, T.; Yamagata, M.; Okamoto, Y.; Akiyama, Y.; Takhashi, H., Kawano, T.; Katayama, Y.; and Niidome, Y. J Controlled Release, 2006, 114, 343-347.
14. Sonavane, G.; Tomoda, K.; Sano, A.; Ohshima, H.; Terada, H.; and Makino, K. Colloids Surf 8, 2008, 65, 1-10.
15. U.S. Pat. No. 5,521,289.
16. Gregori, L.; Hainfeld, J. F.; Simon, M. N.; Goldgaber, D. J. Biol. Chem., 1997, 272, 58-62.
17. Zhu, J.; Lines, B. M.; Ganton, M. D.; Kerr, M. A.; and Workentin, M. S. J. Org. Chem., 2008, 73, 1099-1105. (b) Zhu, J.; Ganton, M. D.; Kerr, M. A.; and Workentin, M. S. J. Am. Chem. Soc., 2007, 129, 4904-4905. (c) Zhu, J., Kell, A. J.; and Workentin, M. S. Org. Lett., 2006, 8, 4993-4996.
18. Ba, H.; Rodriguez-Fernandez, J.; Stefani, F. D.; and Feldmann, J Nano Letters, 2010, 10, 3006-3012.
19. Oh, E.; Susumu, K.; Blanco-Canosa, J. B.; Medintz, I. L.; Dawson, P. E.; and Mattoussi H. Small, 2010, 6, 1273-1278.
20. Brust, M.; Walker, M.; Bethell, D.; Schiffrin, D. J.; and Whyman, R. J. J. Chem. Soc. Chem. Commun., 1994, 801-802.
21. (a) Badia, A; Cuccia, L.; Demers, L.; Morin, F; Lennox, R. B.; and Reven, L. J. Am. Chem. Soc., 1997, 119, 11104-11105. (b) Badia, A ; Singh, S.; Demers, L.; Cuccia, L.; Brown, G. R.; and Lennox, R. B. Chem. Eur J. 1996, 2, 359-363. (c) Badia, A.; Lennox, R. B.; Reven, L. Acc. Chem. Res. 2000, 33, 475-481.
22. (a) Terrill, R. H.; Postlethwaite, T. A.; Chen, C. H.; Poon, C. D.; Terzis, A.; Chen, A. D.; Hutchison, J. E.; Clark, M. R.; Wignall, G.; Londono, J. D.; Superfine, R.; Falvo, M.; Johnson, C. S., Samulski, E. T.; and Murray R. W. J. Am. Chem. Soc., 1995, 117, 12537-12548. (b) Templeton, A. C.: Wuelfing, W. P.; Murray, R. W. Acc. Chem. Res., 2000, 33, 27-36.
23. (a) Whetten, R. L.; Khoury, J. T.; Alvarez, M. M.; Murthy, S.; Vezmar, I.; Wang, Z.; Stephens, P. W. Adv. Mater., 1996, 8, 428-433. (b) Cleveland, C. L.; Landman, U.; Schaaff, T. G.; Shafigullin, M. N.; Stephens, P. W.; Whetten, R. L. Phys. Rev. Lett. 1997, 79, 1873-1876. (c) Whetten, R. L.; Shafigullin, M. N.; Khoury, J. T.; Schaaff, T. G.; Vezmar, I.; Alvarez, M. M.; Wilkinson, A. Acc. Chem. Res., 1999, 32, 397-406.
24. Liu, X., Atwater, M., Wang, J., and Huo, Q. Colloids Surf B, 2007, 58, 3-7.
25. (a) Mayilo, S.; Kloster, M. A.; Wunderlich, M.; Lutich, A.; Klar, T. A.; Nichtl, A.; Kürzinger, K.; Stefani, F. D.; and Feldmann, J. Nano Lett. 2009, 9, 94558-4563. (b) Schneider, G.; and Decher, G. Nano Lett, 2006, 6, 530-536. (c) Hong, R.; Fernandez, J. M.; Nakade, H.; Arvizo, R.; Emrick, T.; and Rotello, V. M. Chem. Commun., 2006, 2347-2349.
26. Templeton, A. C.; Hostetler, M. J.; Kraft, C. J.; Murray, R. W. J Am Chem Soc., 1998, 120, 1906-1911.
27. Wangler, B.; Quandt, G.; Iovkova, L.; Schirrmacher, E.: Wängler, C.: Boenig, G.; Hacker, M.; Schmoeckel, M.; Jurkschat, K.; Bartenstein, P., and Schirrmacher, R. Bioconjugate Chemistry, 2009, 20, 317-321.
28. Guerrero, S.; Herance, J. R.; Rojas, S.; Mena, J. F.; Gispert, J. D., Acosta, G. A.; Albericio, F.; Kogan, M. J. Bioconjugate Chemistry, 2012, 23, 399-408.

1. A maleimide-functionalized gold nanoparticle comprising a ligand monolayer, wherein said ligand monolayer includes at least a PEG-thiolated ligand.

2. The maleimide-functionalized gold nanoparticle of claim 1 having the Formula I: 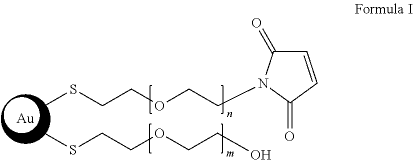
wherein “n” and “m” are integers independently ranging from 1 to 100.

3. The maleimide-functionalized gold nanoparticle of claim 1 having the Formula II: 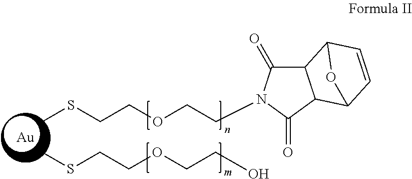
wherein “n” and “m” are integers independently ranging from 1 to 100.

4. A maleimide-functionalized gold nanoparticle-based bioprobe of Formula III: 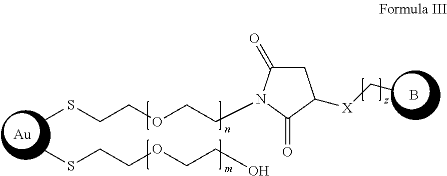
wherein:
“n” and “m” are integers independently ranging from 1 to 100;
X is selected from the group consisting of NR¹ and S;
R¹ is H or C₁₋₆alkyl;
B is a probe, a therapeutic agent or a radioimmaging agent or its precursor; and
“z” is an integer ranging from 0 to 5.

5. The maleimide-functionalized gold nanoparticle-based bioprobe of claim 4, wherein the probe is a biomolecular probe.

6. The maleimide-functionalized gold nanoparticle of claim 1 comprising from about 950 to about 2300 gold atoms.

7. The maleimide-functionalized gold nanoparticle of claim 6 comprising about 1000 gold atoms.

8. The maleimide-functionalized gold nanoparticle of claim 1 comprising a gold core having an average size ranging from about 3 to 4 nm.

9. The maleimide-functionalized gold nanoparticle of claim 8 comprising a gold core having an average size of about 3.2 nm.

10. The maleimide-functionalized gold nanoparticle of claim 2 wherein the maleimide-terminated ligands and PEG ligands are present in a ratio ranging from 1:1 to 1:3.

11. The maleimide-functionalized gold nanoparticle of claim 1 wherein the gold nanoparticle is water-soluble.

12. A process for preparing a maleimide-functionalized gold nanoparticle in accordance with claim 3, the process comprising reacting a PEGylated AuNP with a furan-protected maleimide-PEG-thiol under conditions suitable to provide the maleimide-functionalized gold nanoparticle in accordance with claim 3.

13. A process for preparing a maleimide-functionalized gold nanoparticle of claim 2, the process comprising reacting a PEGylated AuNP with a furan-protected maleimide-PEG-thiol to produce a furan-protected maleimide-functionalized gold nanoparticle of Formula II, followed by heating the furan-protected maleimide-functionalized gold nanoparticle of Formula II under conditions suitable for removal of the furan-protection group and to provide the maleimide-functionalized gold nanoparticle of claim 2.

14. A kit comprising a maleimide-functionalized gold nanoparticle in accordance with claim 4.

15. (canceled)

16. A method for delivering a therapeutic agent to a subject, the method comprising:
a) providing a nanoparticle of Formula I;
b) coupling the therapeutic agent to the nanoparticle of Formula I to form a nanoparticle-drug conjugate; and
c) administering to the subject the nanoparticle-drug conjugate.

17. A method for delivering a radioimmaging agent to a subject, the method comprising:
a) providing a nanoparticle of Formula I;
b) coupling the radioimmaging agent to the nanoparticle of Formula I to form a nanoparticle-radioimmaging agent conjugate; and
c) administering to the subject the nanoparticle-radioimmaging agent conjugate.

18. A radiolabeled maleimide-functionalized gold nanoparticle of Formula IV: 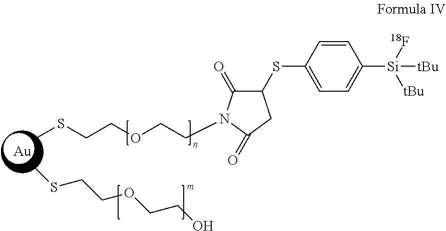
wherein “n” and “m” are integers independently ranging from 1 to 100.

19-21. (canceled)

22. A method of using the radiolabeled maleimide-functionalized gold nanoparticle of Formula IV as a radiotracer for radioimmaging applications, the method comprising administering the radiolabeled maleimide-functionalized gold nanoparticle of Formula IV to a subject.

23. The method of claim 22, wherein the radioimmaging application is PET.

24. A furan protected maleimide-PEG-thiol having the formula: 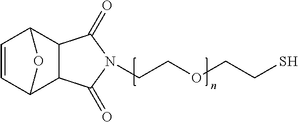
wherein “n” is an integer ranging from 1 to 100.
